(19) United States
(12) Patent Application Publication
(10) Pub. No.: US 2020/0030018 A1
(43) Pub. Date: Jan. 30, 2020

(54) FLEXIBLE CRYOGENIC PROBE TIP

(71) Applicant: Endocare, Inc., Austin, TX (US)

(72) Inventors: John M. Baust, Owego, NY (US); Claudia Lueckge, L'Ile-Bizard (CA); Roy Cheeks, Harper's Ferry, WV (US); John G. Baust, Candor, NY (US); Anthony T. Robi, Binghamton, NY (US)

(21) Appl. No.: 16/593,914

(22) Filed: Oct. 4, 2019

Related U.S. Application Data
(63) Continuation of application No. 14/715,092, filed on May 18, 2015, now abandoned.
Application No. 14/715,092 is a continuation of application No. 13/077,399, filed on Mar. 31, 2011, now abandoned.

(51) Int. Cl. A61B 18/02

(52) U.S. Cl. CPC A61B 18/02; A61B 2017/003

(57) ABSTRACT

An ablation instrument having a longitudinal body with a plurality of sidewalls that form a flexible sleeve, where the plurality of sidewalls that form the flexible sleeve are slotless. The longitudinal body has a proximal end, a distal end, and a central axis. A luminary space is formed within the plurality of sidewalls and at least one internal component is inserted through the proximal end of the longitudinal body and extends through the luminary space to the distal end. The internal component is interconnected with a deflection mechanism for controlling the distal end of the longitudinal body such that the distal end is capable of multi-planar movement.

### RELATED APPLICATIONS

This application is a continuation of U.S. application Ser. No. 14/715,092, filed on May 18, 2015, pending, which is a continuation of U.S. application Ser. No. 13/077,399, filed on Mar. 31, 2011, abandoned, which is claims priority to U.S. Provisional Application No. 61/319,525, filed on Mar. 31, 2010. The above-referenced applications are hereby incorporated by reference in their entirety for all purposes.

### FIELD OF THE INVENTION

The present invention relates generally to the medical treatment technology field and, in particular, to a device for use in cryo-therapeutic procedures.

### BACKGROUND OF THE INVENTION

Cryotherapy is an effective yet minimally invasive alternative to radical surgery and radiation therapy. In this minimally invasive procedure, the destructive forces of freezing are utilized to ablate unwanted tissue in a way that decreases hospitalization time, reduces postoperative morbidity, decreases return interval to daily activities, and reduces overall treatment cost compared to conventional treatments.

Cryosurgery has been shown to be an effective therapy for a wide range of tumor ablation as well as its use to treat atrial fibrillation. Since the early 1960s, treatment of tumors and unwanted tissue has developed around freezing techniques and new instrumentation and imaging techniques to control the procedure. As a result, the complications of cryoablation have been reduced and the efficacy of the technique has increased.

Improved developments in cryoablation instrumentation have led to the advancement in using cryogenic medical devices. The cryogenic medical devices have been designed to deliver subcooled liquid cryogen to various configurations of cryoprobes for the treatment of damaged, diseased, cancerous or other unwanted tissues. The closed or semi-closed systems allow various cryogens to be contained in both the supply and return stages.

Recently, instrumentation has been discovered to convert liquid nitrogen to supercritical nitrogen (SCN) in a cylinder/cartridge cooled by atmospheric liquid nitrogen (−196° C.), the SCN of which can be subcooled and tuned to the liquid phase, attaining an excess temperature. When the SCN is injected into one or more flexible cryoprobes, the SCN flows with minimal friction to the tip of the probe. In the tip, SCN pressure drops due to an increased volume and outflow restriction, heat is absorbed (nucleate boiling) along the inner surface of the tip, micro bubbles of nitrogen gas condense back into a liquid, and the warmed SCN reverts to pressurized liquid nitrogen as it exits the return tube and resupplies the dewar containing atmospheric liquid nitrogen. This flow dynamic occurs within a few seconds, typically in the order of 1 to 10 seconds depending on the probe or attachment configuration, and is regulated by a high-pressure solenoid valve. Once instruments are in place, the cryosurgical procedure can be performed with freeze times in ranges of about 15 seconds to 5 minutes (or ranges thereof), a drastic improvement over current known methods.

Current surgical probes are made of rigid metal materials. If the probes are to be bent or curved, the shape must be pre-formed by tooling and/or bent manually prior to introduction of the probe into the site of treatment in a patient's body. Current probe designs do not address real-time shaping or steering of a probe while at the site of treatment or by any internal control mechanisms. As such, the procedures to date cannot provide a minimally invasive treatment or time effective treatment option since the probes must be manually shaped and positioned repeatedly (with entry and withdrawal from a surgical cavity/opening) to achieve the proper placement.

There exists a need for flexible probes in the surgical ablation field of medicine, specifically in cryo-therapeutic procedures. The flexible cryogenic probes would provide a highly flexible probe tip which can be shaped and steered for proper positioning inside a patient's body. The flexible probe tips would desirably have integrated deflection mechanisms to allow for precise placement of the probes in a minimally invasive manner. In addition, the flexible probes would be capable of being miniaturized so that various cryo-procedures can implement the use of the flexible tip in a safe manner and for a variety of treatment options in the medical environment. The flexible tip would also be capable of being electronically or computer operated to fine-tune its placement and use in surgical procedures.

### SUMMARY OF THE INVENTION

One embodiment of the invention is a flexible cryogenic probe tip. The flexible probe tip has a linear freeze zone at a distal end of the probe that allows for its placement and precisely controlled movements. The flexible cryogenic probe tip conforms to the target tissue surface to create a linear lesion. In addition, the probe tip is steerable to facilitate proper placement with minimal access points into a patient's body. Various configurations of the flexible probe tip, however, allow it to conform and ablate tissue of many sizes, shapes, and/or dimensions.

One embodiment of the ablation instrument comprises: a longitudinal body having one or more sidewalls which form a flexible sleeve, the longitudinal body having a proximal end, a distal end, and a central axis; a luminary space formed within the flexible sleeve; and at least one internal component inserted through the proximal end of the longitudinal body and extending through the luminary space to the distal end; wherein the internal component is interconnected with a deflection mechanism for controlling the distal end of the longitudinal body such that the distal end is capable of multi-planar movement.

In another embodiment, the ablation instrument has a distal end of the longitudinal body that is closed while the proximal end has an open configuration. The internal component can be a deflection wire interconnected with the distal end of the longitudinal body or in connection with any portion of the longitudinal body for control and mobility of the body. In one aspect, the deflection wire is integral with a sidewall of the longitudinal body.

Another embodiment of the invention utilizes a distal end as a flexible linear freeze zone comprising one or more cryolines positioned within the luminary space. The internal component as integrated with the deflection mechanism flexibly positions the distal end within the range of about 0° to about 90° away from the central axis. In addition, the distal end is capable of movement 360° movement about the central axis of the longitudinal body.

Multiple internal components may include a plurality of deflection wires, a manual pull-wire, a pulley or gear system, an electronic or a motorized component, or a wire having electrical response properties, utilized alone or in combination to effect movement. Any number and combination of internal components may be utilized to effect greater mobility. In one aspect, the wire which has electrical response properties comprises shape memory alloys to facilitate contraction or expansion of the wire.

Another embodiment also comprises integrated temperature sensors, electrical monitors, optical visualization materials, or other sensing devices.

In yet another embodiment, the longitudinal body comprises segmented portions, including a multi-segment distal end. In one aspect, the multi-segment distal end comprises a plurality of the internal components anchored upon the one or more sidewalls. In another aspect, the distal end loops back toward the central axis to form a polygonal shape or lasso.

Further, embodiments of the invention have internal components and deflection mechanisms that are cryo-compatible. The internal component, the deflection mechanisms, and the parts and components of the ablation instrument are compatible with supercritical nitrogen.

An embodiment of the invention includes a method of utilizing a flexible probe tip, comprising the steps of: providing an ablation instrument comprising: a longitudinal body having one or more sidewalls which form a flexible sleeve, the longitudinal body having a proximal end, a distal end, and a central axis; a luminary space formed within the flexible sleeve; and at least one internal component inserted through the proximal end of the longitudinal body and extending through the luminary space to the distal end; wherein the internal component is interconnected with a deflection mechanism for controlling the distal end of the longitudinal body such that the distal end is capable of multi-planar movement; positioning the distal end at a tissue site for at least one ablative procedure; and flexibly maneuvering the distal end to precisely treat the tissue site.

A method of the invention also utilizes an ablation instrument by positioning the distal end at a tissue site for at least one ablative procedure; maneuvering the distal end to the tissue site; directing a cryogen from the supply source to the distal end at a pressure above the critical pressure and a temperature below the critical temperature for the cryogen; controlling a flow of cryogen from the supply source to the distal end and back to the supply source; and segmenting control of the distal end mechanically or through the step of controlling the flow of cryogen. In one embodiment, the pressure is reduced within the distal end and through the return portion back to the supply source or vented.

In one embodiment, the ablative procedure is a cryo-treatment. One cryo-treatment includes a step of encircling one or more vessels with the distal end. Another embodiment includes multiple ablative procedures being performed at multiple tissue sites. Further, a step of visualizing placement of the distal end and flexibly positioning the distal end at the tissue site, may include visualization techniques of MRI, X-ray, or optically integrated cameras, alone or in combination.

Various embodiments of the ablative instrument and its method of use include cryo-treatments for cardiovascular (endocardial and epicardial) and cardiac tissue, prostate, kidney, liver, lung, bone, esophageal, pancreatic lymphatic, vascular disease, uterine cancer, fibroids, breast, among others. Any tissue or solid tumor, or growth (benign or cancerous) can be treated. The flexible probe may also be used to target and destroy fat cells as an alternative to liposuction (fat reduction).

### BRIEF DESCRIPTION OF THE DRAWINGS

The invention is best understood from the following detailed description when read with the accompanying drawing figures. It is emphasized that the various features are not necessarily drawn to scale. In fact, the dimensions may be arbitrarily increased or decreased for clarity of discussion.

FIG. 1 is a perspective view of an illustrative embodiment of the device.

FIG. 2 is a cross-sectional view of an illustrative embodiment of the device from FIG. 1 cut across the A-A axis.

### DETAILED DESCRIPTION

In the following detailed description, for purposes of explanation and not limitation, exemplary embodiments disclosing specific details are set forth in order to provide a thorough understanding of the present invention. However, it will be apparent to one having ordinary skill in the art that the present invention may be practiced in other embodiments that depart from the specific details disclosed herein. In other instances, detailed descriptions of well-known devices and methods may be omitted so as not to obscure the description of the present invention.

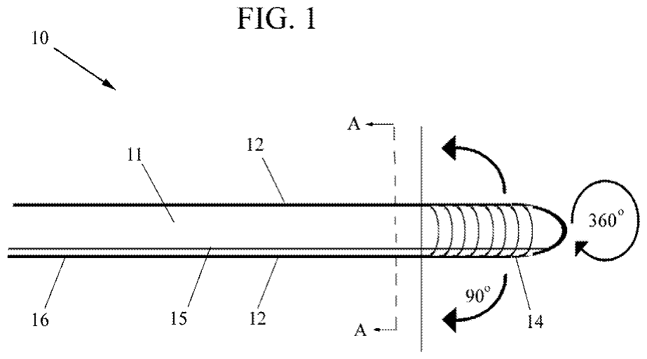

A perspective sideview of a flexible cryogenic ablation probe 10 in accordance with one embodiment of the present invention is illustrated in FIG. 1. The flexible probe 10 is a device 10 that comprises a body shaft 11 which is formed from sidewalls 12, a distal end 14 and proximal end 16. The distal end 14 is a highly flexible linear freeze zone and preferably integral with the body shaft 11. The distal end or tip 14 may vary from about 0.5 cm to about 20 cm in length, scaled according to the size of the probe or catheter shaft or hose and depending upon the use of the probe 10 and treatment procedure. Various modifications of size and shape of the distal end in combination with the probe or catheter utilized can provide numerous treatment options from cancer cryosurgery and treatment of irregular tissue to treatment of heart arrhythmias. In one aspect, the tip section 14 is preferably radio-opaque to enable its visualization in real-time, such as with x-ray, ultrasound, fluoroscopy, or other imaging modality. One or more marks (not shown) on the body of the probe 10 can be visualized and individually identified for treatment selection and procedure, probe location and depth, the placement of the probe and calculation of treatment duration and/or treatment interval corresponding with the size and dimensions of the tissue being treated. In another aspect, the proximal end 16 of the probe 10 is attached to a handle (not shown) which interconnects with the components of the probe to allow for its versatility and ease of use. One such embodiment would include a wire or control integrated with the deflection wire 15 to easily manipulate the movement, extension and flexing of the distal tip 14.

In one embodiment, the probe 10 is comprised of flexible plastic and malleable metal compositions of parts and components to allow it to be steerable in situ. A deflection wire 15 allows for the probe's precise positioning and placement within a patient's body. The flexible probe 10 facilitates minimally invasive access for treatment of diseased tissue, such as in the surgical or electrophysiological treatment of atrial fibrillation (i.e. epi-cardial and endo-cardial treatment options). As illustrated in FIG. 1, the distal tip 14 is rotatable about the central axis of the body 11. The orientation of the probe can be controlled via a steering or deflection mechanism that interconnects with the deflection wire 15. The 360° movement of the distal end 14 allows for a deflection 90° above or below the central axis of the body 11.

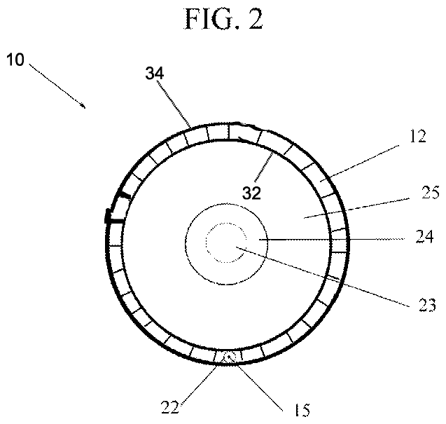

As shown in the embodiment of FIG. 2, the flexible probe 10 integrates a supply line 24, return line 23, and vacuum insulation 25 for cryoablation procedures. The deflection wire 15 is sandwiched in the luminary space 22 between sidewalls 12, where in some embodiments, the sidewalls 12 comprise an inner wall 32 and an outer wall 34 and the luminary space 22 is located between the inner wall 32 and the outer wall 34. In another embodiment, the probe 10 has integrated temperature and electrical monitors and/or sensors. In addition, various embodiments allow for the flexible tip to be compatible with various liquids, gases, and supercritical cryogens.

In an exemplary embodiment, multiple deflection wires of varying lengths may extend through to the distal end of the probe. Multiple deflection points and junctions may allow for a multi-segment distal end of the probe, each segment controllable by a different deflection/steering wire. In one embodiment, the distal end is a bi-directional multi-planar deflection tip having multiple steering wires anchored at various points in the sidewall 12. An individual anchoring point, however, would also be useful such that multiple deflection wires extend at different lengths therefrom. Any number of wires, however, may also be positioned or anchored to any fixation point within the internal vacuum space 25, supply line 24, or return line 23. By integrating the segmentation, the movement capabilities of the distal end allow it to create any number of shapes or achieve positions that are desirable. In one embodiment, the distal end loops back around itself to form a polygonal shape or lasso formation, such as may be desirable in looping the cryo-segment around a vein or artery. Various shapes of the distal end may include an S formation, J or U shape, circle, or any number or combinations of such arrangements, alone or in combination with other probes. The deflection of the probe tip in three-dimensions along the X, Y, and Z planes, and/or in a rotational configuration, provides for various treatment positions and treatment angles.

The mechanism for moving the deflection wire may be by manual wire shortening, such as a pulley or gear system; electronic or motor operation; and/or use of wires with electrical response properties to facilitate contraction or expansion of the wire to effect movement. In one embodiment, Ni—Ti alloys (nickel and titanium alloys, including nitinol which may encompass Co—Ni—Ti alloys) and such compositions are utilized to effect movement. As a shape memory alloy, nitanol has superelasticity to bend and flex as if a biological muscle fiber, and having the bio/physiological and chemical compatibility with the internal human body. Ni—Ti memory alloys can be utilized similar to flex-wires and/or flex-tubes comprising bio-metals.

In another aspect, other metallic compositions, including plastics, aluminum, and copper make the probe MR (magnetic resonance) compatible. Any metal or plastic compositions that are cryo-compatible may be utilized to structure and design a flexible probe tip. In addition, approaches using micro-motors, pneumatics, hydrolics, and/or electric to generate movement may be implemented with the device.

The cryo-probe device may take many forms and be of any size, shape, or dimension. Further, the embodiments of the present invention may be modified to accommodate the size, shape, and dimension of any device or apparatus currently used in the industry. Any number of sensors and/or control mechanisms may also be utilized to facilitate operation of the device at the distal end or throughout the length of the entire probe.

As presented, the multiple embodiments of the present invention offer several improvements over standard cryo-probe devices currently used in the medical industry. The improved cryogenic probes disclosed herein enhance the shaping and steerable functionality of the probe while in use internally within a patient. The integrated deflection mechanism, whether mechanically or electrically controlled allows for precise placement of the probe in a minimally invasive manner. Improvements in the flexible probe design enable easy handling, accessibility, and miniaturization.

The invention being thus described, it would be obvious that the same may be varied in many ways by one of ordinary skill in the art having had the benefit of the present disclosure. Such variations are not regarded as a departure from the spirit and scope of the invention, and such modifications as would be obvious to one skilled in the art are intended to be included within the scope of the following claims and their legal equivalents.

1. An ablation instrument comprising:
a longitudinal body having one or more sidewalls which form a flexible sleeve, the longitudinal body having a proximal end, a distal end, and a central axis;
a luminary space formed within the flexible sleeve; and
at least one internal component inserted through the proximal end of the longitudinal body and extending through the luminary space to the distal end;
wherein the internal component is interconnected with a deflection mechanism for controlling the distal end of the longitudinal body such that the distal end is capable of multi-planar movement.

2. The ablation instrument of claim 1, further comprising multiple internal components including a plurality of deflection wires, a manual pull-wire, a pulley or gear system, an electronic or a motorized component, or an electrical response wire, utilized alone or in combination to effect movement and/or integrated temperature sensors, electrical monitors, optical visualization materials, or other sensing devices.

3. The ablation instrument of claim 2, wherein the electrical response wire comprises shape memory alloys to facilitate contraction or expansion of the electrical response wire.

4. The ablation instrument of claim 1, wherein the longitudinal body comprises segmented portions, including a multi-segment distal end.

5. The ablation instrument of claim 4, wherein the multi-segment distal end comprises a plurality of the internal components anchored upon the one or more sidewalls.

6. A method of utilizing an ablation instrument, comprising the steps of:
providing an ablation instrument according to claim 1:
positioning the distal end at a tissue site for at least one ablative procedure; and
flexibly maneuvering the distal end to precisely treat the tissue site.

7. The method of claim 6, wherein the ablative procedure is a cryo-treatment and the cryo-treatments include applications in cardiac tissue, tumor tissue, vasculature, reproductive tissues and organs, and cosmetic applications.

8. The method of claim 7, wherein the ablative procedure includes a step of encircling one or more vessels with the distal end and/or a step of visualizing placement of the distal end and flexibly positioning the distal end at the tissue site, including visualization techniques of MRI, X-ray, or optically integrated cameras, alone or in combination and/or the step of positioning multiple ablative procedures are performed at multiple tissue sites.

9. The ablation instrument of claim 1 wherein the ablation instrument is a cryo-ablation instrument and the luminary space formed within the flexible sleeve includes a supply line which directs a cryogen from a supply source to the distal end and also including a return portion which provides flow of cryogen back to the supply source.

10. A method of utilizing an ablation instrument, comprising the steps of:
providing an ablation instrument of claim 9;
positioning the distal end at a tissue site for at least one ablative procedure;
maneuvering the distal end to the tissue site;
directing a cryogen from the supply source to the distal end; controlling a flow of cryogen from the supply source to the distal end and back to the supply source; and
segmenting control of the distal end mechanically or through the step of controlling the flow of cryogen.

11. The ablation instrument of claim 1, wherein the plurality of sidewalls that form the flexible sleeve are slotless.

12. The ablation instrument of claim 11, wherein the distal end of the longitudinal body is closed and the proximal end has an open configuration.

13. The ablation instrument of claim 11, wherein the internal component is a deflection wire.

14. The ablation instrument of claim 13, wherein the deflection wire is interconnected with distal end of the longitudinal body.

15. The ablation instrument of claim 13, wherein the deflection wire is integral with a sidewall.

16. The ablation instrument of claim 11, wherein the distal end is a flexible linear freeze zone comprising one or more cryolines positioned within the luminary space.

17. The ablation instrument of claim 11, wherein the internal component as integrated with the deflection mechanism flexibly positions the distal end within the range of about 0° to about 90° away from the central axis.

18. The ablation instrument of claim 17, wherein the distal end is capable of 360° movement about the central axis of the longitudinal body.

19. The ablation instrument of claim 11, wherein the longitudinal body comprises segmented portions, including a multi-segment distal end.

20. The ablation instrument of claim 11, wherein the distal end loops back toward the central axis to form a polygonal shape or lasso.

21. The ablation instrument of claim 11, wherein the internal component in combination with the deflection mechanism are cryo-compatible.

22. The ablation instrument of claim 11, wherein the internal component is compatible with supercritical nitrogen.

23. The cryoablation instrument of claim 11 wherein:
the longitudinal body has a sidewall that comprises an inner wall and an outer wall that forms a flexible sleeve, and
the luminary space is formed within the sidewall between the inner wall and the outer wall.

24. The cryoablation instrument of claim 23, wherein the distal end of the longitudinal body is closed and the proximal end has an open configuration.

25. The cryoablation instrument of claim 23, wherein the internal component is a deflection wire.

26. The cryoablation instrument of claim 25, wherein the deflection wire is integral with a sidewall.

27. The cryoablation instrument of claim 23, wherein the internal component as integrated with the deflection mechanism flexibly positions the distal end within the range of about 0° to about 90° away from the central axis.

28. The flexible cryoablation of claim 23, wherein the distal end is capable of 360° movement about the central axis of the longitudinal body.

29. The flexible cryoablation instrument of claim 23, wherein the luminary space includes a supply line that directs a cryogen from a supply source to the distal end and a return portion that provides flow of cryogen back to the supply source.

30. A method of utilizing an ablation instrument, comprising the steps of:
providing an ablation instrument of claim 11 that further comp a supply line that directs a cryogen from a supply source to the distal end and a return portion that provides a flow of cryogen back to the supply source; and
positioning the distal end at the tissue site;
directing a cryogen from the supply source to the distal end;
controlling a flow of cryogen from the supply source to the distal end and back to the supply source; and
segmenting control of the distal end mechanically or through the step of controlling the flow of cryogen.
